(19) United States
(12) Patent Application Publication
(10) Pub. No.: US 2024/0052308 A1
(43) Pub. Date: Feb. 15, 2024

(54) HUMAN SCALP HAIR FOLLICLE SINGLE CELL SUSPENSION, PREPARATION METHOD AND APPLICATION THEREOF

(71) Applicant: SHANGHAI ICURE BIOSCIENCE CO., LTD, Shanghai (CN)

(72) Inventors: Yamei SUN, Shanghai (CN); Wen GU, Shanghai (CN); Shaohua MA, Shanghai (CN); Shuling NIE, Shanghai (CN)

(73) Assignee: SHANGHAI ICURE BIOSCIENCE CO., LTD, Shanghai (CN)

(21) Appl. No.: 18/259,588

(22) PCT Filed: May 7, 2021
(86) PCT No.: PCT/CN2021/091999

§ 371 Date: Jun. 28, 2023

(30) Foreign Application Priority Data
Dec. 28, 2020 (CN) 202011575524.5

(51) Int. Cl. C12N 5/071

(52) U.S. Cl. CPC C12N 5/0626; C12N 2509/00

(57) ABSTRACT

A preparation method of a human scalp hair follicle single cell suspension includes the following steps: step 1, a fresh isolated scalp tissue is pretreated with a digestive enzyme solution in a low temperature environment; step 2, the hair follicles can be effectively dissociated by digestion with the digestive enzyme solution at 30-37° C.; step 3, the dissociated hair follicles are digested with pancreatin-EDTA to obtain a human scalp hair follicles single cell suspension. In addition, the invention discloses a human scalp hair follicle single cell suspension prepared by the above method and use thereof. The method of the present invention is short in time and the hair follicles dissociated are high in integrity, and the subsequent isolated single cell populations are large in number and high in vitality, which solves the problems of incomplete separation of hair follicles from isolated scalp tissue, long cell extraction time, and low cell viability.

### CROSS REFERENCE TO THE RELATED APPLICATIONS

This application is the national phase entry of International Application No. PCT/CN2021/091999, filed on May 7, 2021, which is based upon and claims priority to Chinese Patent Application No. 202011575524.5, filed on Dec. 28, 2020, the entire contents of which are incorporated herein by reference.

### TECHNICAL FIELD

The invention relates to the field of biotechnology, in particular, to a human scalp hair follicle single cell suspension and preparation method and application thereof.

### BACKGROUND

Vitiligo is a kind of skin disease caused by skin fading due to a lack of epidermal melanin and the main symptoms are white patches appearing on the skin. The disease is common, and with an estimated prevalence of 1% to 2% in the population. Due to the acceleration pace of social life, the aggravation of people's life and work pressure, the intensification of environmental pollution, and the change of dietary structure, etc., the incidence of vitiligo has been showing an upward trend. Vitiligo has great damage to the appearance of patients, and the treatment is difficult, which brings great obstacles to the work, study, employment, mate selection and social interaction activities of patients. Vitiligo patients often have a tendency to lower self-esteem or depression. Therefore, the treatment of vitiligo patients is a process of helping patients regain confidence and restore social interaction, which has important social significance.

Currently, there are many treatments for vitiligo, such as topical or systemic use of glucocorticoids, topical calcineurin inhibitors or vitamin D3 derivatives, narrow-wave ultraviolet phototherapy, excimer laser, traditional Chinese medicine, etc., but because each method has its own limitations, such as inaccurate efficacy, side effects of drugs, and cancer-causing risk of ultraviolet rays, etc., which eventually lead to incomplete recoloration of vitiligo. Autologous melanocyte transplantation is another effective treatment method and autologous epidermal cell transplantation is the most used method; however, due to the scarring of the donor area and the low content of epidermal melanocytes, the treatment area is limited and the recoloration is uneven, which can not achieve a satisfactory curative effect.

Human scalp hair follicles are rich in melanocytes, and the ratio of melanocytes to epithelial cells in human hair follicles is 1:5 (Cichorek M et al, 2013); while the ratio of melanocytes to epithelial cells in human skin is 1:36 (Hoath S B and Leahy D G, 2003). Therefore, human hair follicles are a very good source of melanocytes. The use of the extracted hair follicle single cell suspension in the treatment of vitiligo can effectively solve the problem of limited treatment area caused by insufficient melanocytes and can also effectively solve the problem of uneven recoloration. In addition, the scalp, as a donor area, is covered by hair and will not produce obvious scars on the appearance, and easy to be accepted by patients. Therefore, an efficient and rapid preparation of high-quality human scalp hair follicle single cell suspension is a key step in the treatment of vitiligo with autologous melanocyte transplantation. However, the traditional laboratory techniques of isolating hair follicles and extracting cells often require a long time (overnight treatment) to process isolated scalp samples, which is time-consuming and can easily lead to a decline in cell viability, limiting its clinical application.

Therefore, there is an urgent need to develop a new rapid and efficient method for preparing human scalp hair follicle single cell suspension.

### SUMMARY

A technical problem to be solved by the present invention is to provide an efficient and rapid method for preparing a human scalp hair follicle single cell suspension, which solves the problems of incomplete separation of hair follicles from isolated scalp tissue blocks, long cell extraction time, and low cell number and viability, and lay a certain foundation for the subsequent application of cell transplantation.

A second technical problem to be solved by the present invention is the human scalp hair follicle single cell suspension prepared by the above method.

A third technical problem to be solved by the present invention is to provide an application of the human scalp hair follicle single cell suspension.

In one aspect, the present invention provides a preparation method of a human scalp hair follicle single cell suspension, comprising the following steps:
step 1, a fresh isolated scalp tissue is pretreated with a digestive enzyme solution in a low temperature environment;
step 2, the hair follicles can be effectively dissociated by digestion with the digestive enzyme solution at 30-37° C.;
step 3, the dissociated hair follicles are digested with pancreatin-EDTA to obtain a human scalp hair follicle single cell suspension.

As a preferred technical solution of the present invention, in step 1, the low temperature environment is 2-8° C.; and the treatment time of the pretreatment with the digestive enzyme solution is 1-1.5 hours.

As a preferred technical solution of the present invention, in step 1 and step 2, a mass percent concentration of the digestive enzyme solution is 0.5-1.5%; the digestive enzyme solution is prepared according to the following formula: a dispase solution with a mass percentage concentration of 1%-3% and a collagenase solution with a mass percentage concentration of 1%-3% are mixed evenly in equal volumes to obtain the digestive enzyme solution.

As a preferred technical solution of the present invention, the following steps are added before step 1: the following steps are added before step 1: step (1), a human scalp pretreatment: in a sterile environment, excess fat is cut off from the isolated scalp tissue block, and cut into blocks with a size of 0.1-0.2 cm2, this step takes 3-5 minutes; step (2), a disinfection treatment is carried out, and the disinfection treatment time is 9-21 minutes.

As a preferred technical solution of the present invention, the disinfection treatment is specifically: lumps of scalp tissue are placed in PBS with antibiotics, washed twice for 5 minutes each time, and finally washed once with PBS without antibiotics for 5 minutes; the PBS with antibiotics comprises 400 u/ml penicillin, 400 ug/ml streptomycin, and 10 ug/ml amphotericin B.

As a preferred technical solution of the present invention, step 1 is specifically: a pre-cooled 0.5-1.5% digestive enzyme solution is added into a clean and sterile tube, and the isolated scalp tissue is submerged in it, if there is too much scalp tissue, the volume of digestive enzyme solution should be subject to completely submerge the tissue, and stand at 2-8° C. for 1-1.5 hours.

As a preferred technical solution of the present invention, step 2 is specifically: the digestive enzyme solution is incubated and digested with shaking at 100-180 rpm at 30-37° C. for 30 minutes to 1 hour, and the tissue blocks are transferred to a culture dish with DMEM to dissociate hair follicles.

As a preferred technical solution of the present invention, the digestion time in step 3 is 15-30 minutes, and step 3 is specifically: the dissociated hair follicles are transferred to 0.25%-0.5% pancreatin-EDTA, the hair follicles are completely submerged in it, stand for digestion for 15-30 minutes at 30-37° C., then DMEM and human serum or fetal bovine serum are added to make up the volume to 5-10 ml, wherein a final concentration of the human serum or fetal bovine serum is 5-10%, mixed well and then pipetted, the number of pipetting should not exceed 30 times; the hair rod are removed, the remaining liquid is centrifuged at 800-1200 rpm for 5-10 minutes at room temperature, then the supernatant is removed, and the pellet is resuspended with DMEM containing 5-10% human serum or fetal bovine serum to obtain hair follicles single cell suspension.

In a second aspect, the present invention provides a human scalp hair follicle single cell suspension prepared by the above method.

In a third aspect, the present invention provides an application of the human scalp hair follicle single cell suspension for the preparation of a medicament to treat vitiligo.

The terminology in the text is explained as follows:
the dispase solution refers to: a certain amount of dispase solid powder is dissolved in a certain volume of DMEM, and after being completely dissolved, it is prepared into a dispase solution with a certain mass percentage concentration;
the collagenase solution refers to: a certain amount of collagenase solid powder is dissolved in a certain volume of DMEM, and after being completely dissolved, it is prepared into a collagenase solution with a certain mass percentage concentration;
the digestive enzyme solution refers to: a mixture of the above two dispase solution and collagenase solution in a certain amount volume;
the human scalp hair follicle refers to: a sheathing structure surrounding the hair root of a human being, which is divided into an inner hair root sheath, an outer hair root sheath, and a fiber sheath from inside to outside; the hair follicle is located in the dermis and subcutaneous tissue, and can be divided into 3 parts, the hair follicle funnel, the isthmus of the hair follicle and the lower part of the hair follicle;
the single cell suspension refers to: digesting the hair follicle tissue, and separating cells from the hair follicle tissue into a single, free cell group;
the pancreatin-EDTA refers to: 0.25 g pancreatin powder and 0.02 g EDTA are completely dissolved in a 100 ml PBS solution, that is, a solution containing 0.25% pancreatin-EDTA is prepared;
the culture dish with DMEM refers to: adding a certain amount of DMEM to the cell culture dish;
the human serum refers to: derived from human blood, it is a slightly viscous liquid with light yellow and clear character and appearance, no hemolysis, and no foreign matter; it can be prepared on site, and there are also finished products on sale; serum contains various serum proteins, polypeptides, fats, carbohydrates, growth factors, hormones, inorganic substances, etc., which can promote cell growth;
the fetal bovine serum refers to: derived from fetal bovine, it is a slightly viscous liquid with light yellow and clear character and appearance, no hemolysis, and no foreign matter, and there are also finished products on sale; the serum contains various serum proteins, polypeptides, fats, carbohydrates, growth factors, hormones, inorganic substances, etc., which can promote cell growth.

Compared with the prior art, the present invention has the following beneficial effects:
in the present invention, 0.5-1.5% digestive enzyme solution (a mixture of the dispase and the collagenase, hereinafter referred to as a digestive enzyme solution) is used for short-term pretreatment of isolated human scalp tissue at a low temperature (2-8° C.), and then 0.5-1.5% digestive enzyme solution digests the tissue at 30-37° C., separates the hair follicles, and then digests the separated hair follicles with pancreatin-EDTA to prepare a human scalp hair follicle single cell suspension with a large number and high vitality. Compared with the conventional laboratory isolation method, the hair follicles dissociated by the present invention method take a short time and have a high integrity, and the number of single cell groups isolated subsequently is large (30 hair follicles, the number of available cells>10⁶), and the vitality is high (the activity of trypan blue staining is ≥80%), which solves the problems of incomplete separation of hair follicles from isolated scalp tissue, long time for cell extraction, and low cell number and validity, which has been laid a certain foundation for the subsequent application of cell transplantation.

### BRIEF DESCRIPTION OF THE DRAWINGS

FIG. 1 is a flow diagram of the preparation method of human scalp hair follicle single cell suspension in Examples 1-3 of the invention.

FIG. 2 is a schematic diagram of microscope observation of hair follicles obtained by the method of the invention in Control Experiment 1.

FIG. 3 a schematic diagram of microscope observation of hair follicle obtained by conventional methods in Control Experiment 1.

FIG. 4 is a schematic diagram of the results of applying human scalp hair follicle single cell suspension prepared by the method of the invention to patients with vitiligo in Clinical Trial 1.

### DETAILED DESCRIPTION OF THE EMBODIMENTS

The following is a further detailed description of the present invention in combination with specific examples and attached drawings. The protection scope of the present invention is not limited to the following examples.

Materials:

Materials used in the following examples and experiments, such as DMEM, PBS, digestive enzyme solution, pancreatin-EDTA solution, human serum, fetal bovine serum, etc., are all commercially available products.

Digestive enzyme solution can be self-configured according to the following formula: 1%-3% dispersible enzyme solution and 1%-3% collagenase solution is mixed evenly in equal volume to obtain a digestive enzyme solution.

Human serum can use the patient's own serum, which is prepared on the site of blood drawing on the day of surgery.

### Example 1

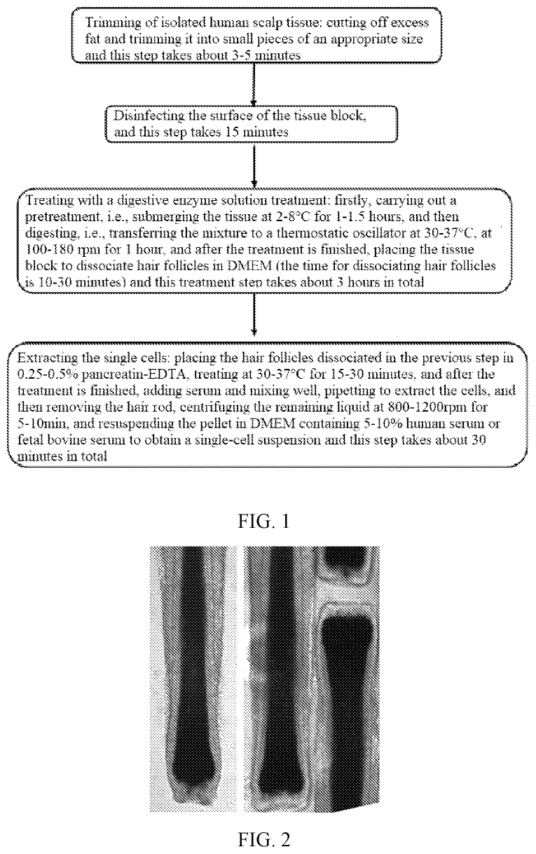

As shown in FIG. 1, the preparation method of a human scalp hair follicle single cell suspension of the present invention comprises the following steps:
1. a biological safety cabinet is sterilized by an ultraviolet radiation for 30 minutes, and then the front door of the biological safety cabinet is opened to a suitable position, the ultraviolet lamp automatically senses the door to open and close, and the internal sterile air circulation system sensing door opens automatically; at this moment, an aseptic operation can be performed;
2. take three clean petri dishes with a diameter of 10 cm, place them in the biological safety cabinet, add 5 ml DMEM respectively, and set them aside for backup;
3. put the pre-prepared clean surgical scissors and tweezers in the biological safety cabinet for backup;
4. place a fresh isolated human scalp tissue in a petri dish with DMEM, trim the block (cut the excess fat off the isolated scalp tissue block, and cut it into pieces about 0.2 cm² in size), this step takes about 3 minutes;
5. disinfection treatment: place the trimmed scalp tissue in PBS with antibiotics (400 u/ml penicillin, 400 ug/ml streptomycin, and 10 ug/ml amphotericin B), wash twice for 5 minutes each time, and finally wash once with PBS without antibiotics for 5 minutes, then put it into 0.5% digestive enzyme solution to completely submerge it (if there is too much scalp tissue, the volume of digestive enzyme solution should be subject to completely submerge the tissue), and stand at 2° C. for 1.5 hours.
6. after soaking, transfer directly to incubate and digest with shaking at 150 rpm for 1 h at 37° C., then transfer the tissue block to a petri dish with DMEM, peel off the epidermis with tweezers, pull out the hair, and place the pulled out hair in another petri dish with DMEM, the hair follicles are attached to the hair and will be pulled out together, and the integrity of the hair follicles will be observed under a microscope;
7. transfer the hair (that is, the separated hair follicle) to 0.25% pancreatin-EDTA solution, make the hair completely submerged in the liquid, then let it stand for 30 minutes at 37° C., and then add DMEM and human serum (if not used in clinical transplantation, fetal bovine serum can be used to replace human serum), to make up the volume to 5 ml, wherein a final concentration of human serum (fetal bovine serum) is 5%, pipet after mixing, the number of pipetting does not exceed 30 times; remove the hair shaft, and the remaining liquid is centrifuged at room temperature at 1000 rpm for 5 minutes, then remove the supernatant, and resuspend the pellet in DMEM containing 5% human serum (fetal bovine serum) to obtain a hair follicle single cell suspension.

### Example 2

As shown in FIG. 1, the preparation method of a human scalp hair follicle single cell suspension of the present invention comprises the following steps:
1. a biological safety cabinet is sterilized by an ultraviolet radiation for 30 minutes, and then the front door of the biological safety cabinet is opened to a suitable position, the ultraviolet lamp automatically senses the door to open and close, and the internal sterile air circulation system sensing door opens automatically; at this moment, an aseptic operation can be performed;
2. take three clean petri dishes with a diameter of 10 cm, place them in the biological safety cabinet, add 5 ml DMEM respectively, and set them aside for backup;
3. put the pre-prepared clean surgical scissors and tweezers in the biological safety cabinet for backup;
4. place a fresh isolated human scalp tissue in a petri dish with DMEM, trim the block (cut the excess fat off the isolated scalp tissue block, and cut it into pieces about 0.1 cm² in size), this step takes about 5 minutes;
5. disinfection treatment: place the trimmed scalp tissue in PBS with antibiotics (400 u/ml penicillin, 400 ug/ml streptomycin, and 10 ug/ml amphotericin B), wash twice for 3 minutes each time, and finally wash once with PBS without antibiotics for 3 minutes, then put it into 1.5% digestive enzyme solution to completely submerge it (if there is too much scalp tissue, the volume of digestive enzyme solution should be subject to completely submerge the tissue), and stand at 8° C. for 1 hours.
6. after soaking, transfer directly to incubate and digest with shaking at 100 rpm for 30 minutes at 30° C., then transfer the tissue block to a petri dish with DMEM, peel off the epidermis with tweezers, pull out the hair, and place the pulled out hair in another petri dish with DMEM, the hair follicles are attached to the hair and will be pulled out together, and the integrity of the hair follicles will be observed under a microscope;
7. transfer the hair (that is, the separated hair follicle) to 0.5% pancreatin-EDTA solution, make the hair completely submerged in the liquid, then let it stand for 15 minutes at 30° C., and then add DMEM and human serum (if not used in clinical transplantation, fetal bovine serum can be used to replace human serum), to make up the volume to 10 ml, wherein a final concentration of human serum (fetal bovine serum) is 10%, pipet after mixing, the number of pipetting does not exceed 30 times; remove the hair shaft, and the remaining liquid is centrifuged at room temperature at 800 rpm for 10 minutes, then remove the supernatant, and resuspend the pellet in DMEM containing 10% human serum (fetal bovine serum) to obtain a hair follicle single cell suspension.

### Example 3

As shown in FIG. 1, the preparation method of a human scalp hair follicle single cell suspension of the present invention comprises the following steps:
1. a biological safety cabinet is sterilized by an ultraviolet radiation for 30 minutes, and then the front door of the biological safety cabinet is opened to a suitable position, the ultraviolet lamp automatically senses the door to open and close, and the internal sterile air circulation system sensing door opens automatically; at this moment, an aseptic operation can be performed;
2. take three clean petri dishes with a diameter of 10 cm, place them in the biological safety cabinet, add 5 ml DMEM respectively, and set them aside for backup;
3. put the pre-prepared clean surgical scissors and tweezers in the biological safety cabinet for backup;
4. place a fresh isolated human scalp tissue in a petri dish with DMEM, trim the block (cut the excess fat off the isolated scalp tissue block, and cut it into pieces about 0.15 cm² in size), this step takes about 4 minutes;
5. disinfection treatment: place the trimmed scalp tissue in PBS with antibiotics (400 u/ml penicillin, 400 ug/ml streptomycin, and 10 ug/ml amphotericin B), wash twice for 7 minutes each time, and finally wash once with PBS without antibiotics for 7 minutes, then put it into 1.0% digestive enzyme solution to completely submerge it (if there is too much scalp tissue, the volume of digestive enzyme solution should be subject to completely submerge the tissue), and stand at 6° C. for 1.2 hours.
6. after soaking, transfer directly to incubate and digest with shaking at 180 rpm for 50 minutes at 35° C., then transfer the tissue block to a petri dish with DMEM, peel off the epidermis with tweezers, pull out the hair, and place the pulled out hair in another petri dish with DMEM, the hair follicles are attached to the hair and will be pulled out together, and the integrity of the hair follicles will be observed under a microscope;
7. transfer the hair (that is, the separated hair follicle) to 0.4% pancreatin-EDTA solution, make the hair completely submerged in the liquid, then let it stand for 20 minutes at 35° C., and then add DMEM and human serum (if not used in clinical transplantation, fetal bovine serum can be used to replace human serum), to make up the volume to 8 ml, wherein a final concentration of human serum (fetal bovine serum) is 7%, pipet after mixing, the number of pipetting does not exceed 30 times; remove the hair shaft, and the remaining liquid is centrifuged at room temperature at 1200 rpm for 7 minutes, then remove the supernatant, and resuspend the pellet in DMEM containing 7% human serum (fetal bovine serum) to obtain a hair follicle single cell suspension.

Control Experiment 1

The hair follicles obtained by the method of the present invention (such as the above Example 3) are shown in FIG. 2. Most of the hair follicles obtained by the method of the present invention are relatively complete, the bulge area is full, and the cells at hair root are relatively complete.

The hair follicles obtained by conventional laboratory preparation methods are shown in FIG. 3, and the cells at the hair root are severely lost.

The specific steps of the conventional laboratory preparation method are as follows:
trimming and disinfection treatment are the same as the present invention, then submerge overnight with 0.5% dispase solution at 2-8° C., then transfer to 30-37° C. for treatment for 1.5 h, treated in 0.5% collagenase solution at 30-37° C. for 1.5 h, then the hair follicles are separated, and the pancreatin treatment is the same as that of the present invention.

Comparing FIG. 2 and FIG. 3, it can be seen that the integrity of the hair follicles obtained by the method of the present invention is much higher than that of the hair follicles obtained by conventional laboratory preparation methods, and an unexpected technical effect compared with the prior art is achieved.

Control Experiment 2

The cell preparation time of the present invention is short: from obtaining the isolated human scalp tissue to the preparation of the single cell suspension, the present invention takes about 4 hours (see FIG. 1, all steps in FIG. 1 add up to about 4 hours), and patients can be discharged from the hospital on the same day of surgery; but the conventional laboratory preparation method (the steps of the conventional laboratory preparation method are the same as the Control Experiment 1) requires overnight treatment, and the single cell suspension cannot be obtained on the same day. It can be seen that the method of the present invention greatly shortens the cell preparation time and achieves unexpected technical effects compared with the prior art.

Control Experiment 3

The single cell suspension obtained by the method of the present invention and the single cell suspension obtained by the conventional laboratory preparation method (the steps of the conventional laboratory preparation method are the same as those of the Control Experiment 1) were used for comparison of the total cell number, viable cell number and cell viability data, see Table 1 for details. Table 1 is the data comparison of the single cell suspension obtained by the above two methods.

TABLE 1

| Experimental | | | | | |
|---|---|---|---|---|---|
| data/ | Scalp | | | | |
| Experimental | area | Hair | Total cell | viable cell | Cell |
| method | (cm²) | number | number | number | viability |
| Conventional method | 0.4 | 54 | 3.66 × 10⁶ | 2.08 × 10⁶ | 57% |
| | 0.4 | 56 | 3.01 × 10⁶ | 1.59 × 10⁶ | 53% |
| Method of the | 0.4 | 64 | 5.32 × 10⁶ | 4.33 × 10⁶ | 81% |
| invention | 0.4 | 61 | 5.55 × 10⁶ | 4.83 × 10⁶ | 87% |

The conventional method in Table 1 was independently tested twice, and the method of the invention was independently tested twice, and the scalp patches were all from vitiligo subjects of clinical trials, and the areas were equal. As the number of hair follicles and hairs of different people is different, the number of hair roots is slightly different. After treatment, the obtained cell suspension is counted by Bio-Rad TC20 cell counter, and the cell viability is counted by trypan blue staining. It can be seen from the experimental results that the single cell population isolated by the method of the invention has a large number (30 hair follicles, the cell number is >106) and high vitality (the cell vitality of the single cell suspension obtained by the method of the invention is more than 80%, which is much higher than the cell vitality of the single cell suspension obtained by the conventional laboratory preparation method 57% and 53%). It can be seen that the cell viability of the single-cell suspension obtained by the method of the present invention has achieved an unexpected technical effect compared with the prior art.

Clinical Trial 1

The human scalp hair follicle single cell suspension obtained by the method of the present invention is applied to patients with vitiligo, the steps are as follows:
1. routine disinfection of the surgical area of patients with vitiligo: administer local anesthesia with 1% lidocaine and 1% epinephrine;
2. use an electric dermabrasion agent to grind the skin surface of the operation area until tiny bleeding spots appear;
3. the human scalp hair follicle single cell suspension obtained by the method of the present invention is dripped on the grinding area, and then immediately cover with polycarbonate dialysis membrane;
4. use vaseline ointment gauze to press and fix to prevent the operation site from being pulled; that is, to complete the transplantation application of the single cell suspension obtained by the method of the present invention on vitiligo.

The transplant is performed by doctors in the operating room, and the steps of the transplant operation are basically adopted in the operating rooms of most hospitals (such as disinfection methods, grinding methods, and fixation methods).

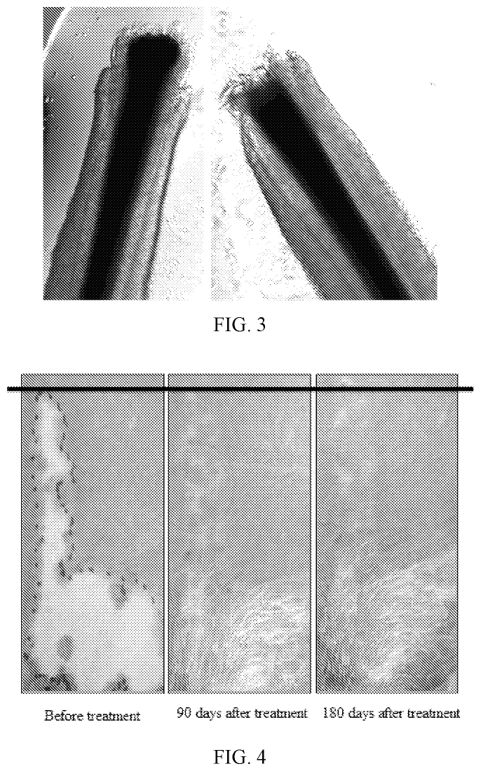

As shown in FIG. 4, it can be seen that the single cell suspension obtained by the method of the present invention is applied to patients with vitiligo, compared with before treatment, the recoloration of the test area in three months (90 days) and six months (180 days) after the operation is even and continuous, achieving an unexpected effect compared with the existing treatment methods; the before and after treatment photos were taken when the patient was followed up at the outpatient doctor's office, and the camera is a Canon IXUS 190 digital camera; when shooting, try to ensure that the outside conditions of the before and after photos are the same.

The above examples are just to illustrate the technical concept and features of the present invention, so that those skilled in the art can understand the content of the present invention and implement it accordingly and cannot limit the scope of protection of the present invention. All equivalent changes and modifications made according to the essence of the content of the present invention shall fall within the protection scope of the present invention.

What is claimed is:

1. A preparation method of a human scalp hair follicle single cell suspension, comprising the following steps:
step 1, pretreating a fresh isolated scalp tissue with a digestive enzyme solution in a low temperature environment to obtain a pretreated scalp tissue;
step 2, effectively dissociating hair follicles of the pretreated scalp tissue by a digestion with the digestive enzyme solution at 30-37° C. to obtain a dissociated hair follicles;
step 3, digesting the dissociated hair follicles with pancreatin-EDTA to obtain the human scalp hair follicle single cell suspension.

2. The preparation method according to claim 1, wherein in step 1, the low temperature environment is 2-8° C.; and a treatment time of the pretreating with the digestive enzyme solution is 1-1.5 hours.

3. The preparation method according to claim 1, wherein in step 1 and step 2, a mass percent concentration of the digestive enzyme solution is 0.5-1.5%; the digestive enzyme solution is prepared according to the following formula: evenly mixing a dispase solution with a mass percentage concentration of 1%-3% and a collagenase solution with a mass percentage concentration of 1%-3% in equal volumes to obtain the digestive enzyme solution.

4. The preparation method according to claim 1, wherein the following steps are added before step 1: substep 1, a human scalp pretreatment: in a sterile environment, cutting off an excess fat from an isolated scalp tissue block to obtain a resulting scalp tissue block, and cutting the resulting scalp tissue block into blocks with a size of 0.1-0.2 cm², and substep takes 3-5 minutes; substep 2, carrying out a disinfection treatment on the blocks to obtain the fresh isolated scalp tissue, and a disinfection treatment time is 9-21 minutes.

5. The preparation method according to claim 4, wherein the disinfection treatment comprises: placing the blocks in PBS with antibiotics, washing the blocks twice for 5 minutes each time, and finally washing the blocks once with PBS without antibiotics for 5 minutes; the PBS with antibiotics comprises 400 u/ml penicillin, 400 ug/ml streptomycin, and 10 ug/ml amphotericin B.

6. The preparation method according to claim 1, wherein step 1 comprises: adding a pre-cooled 0.5-1.5% digestive enzyme solution into a clean and sterile tube to submerge the fresh isolated scalp tissue, wherein if the fresh isolated scalp tissue is too much, a volume of the pre-cooled 0.5-1.5% digestive enzyme solution is subject to completely submerge the fresh isolated scalp tissue, and standing the pre-cooled 0.5-1.5% digestive enzyme solution submerging the fresh isolated scalp tissue at 2-8° C. for 1-1.5 hours.

7. The preparation method according to claim 1, wherein step 2 comprises: incubating and digesting the digestive enzyme solution by shaking at 100-180 rpm at 30-37° C. for 30 minutes to 1 hour, and the pretreated scalp tissue are transferred to a culture dish with DMEM to dissociate the hair follicles.

8. The preparation method according to claim 1, wherein a digestion time in step 3 is 15-30 minutes, and step 3 comprises: transferring the dissociated hair follicles to 0.25%-0.5% pancreatin-EDTA to completely submerge the dissociated hair follicles, standing the 0.25%-0.5% pancreatin-EDTA submerging the dissociated hair follicles for the digesting for 15-30 minutes at 30-37° C. to obtain a digested solution, then adding DMEM and a human serum or a fetal bovine serum in the digested solution to make up a volume of a resulting mixture to 5-10 ml, wherein a final concentration of the human serum or the fetal bovine serum is 5-10%, mixing the resulting mixture well and then pipetting the resulting mixture, a number of the pipetting does not exceed 30 times; removing a hair rod from the resulting mixture to obtain a remaining liquid, centrifuging the remaining liquid at 800-1200 rpm for 5-10 minutes at room temperature to obtain a centrifuged remaining liquid, and then removing a supernatant from the centrifuged remaining liquid to obtain a pellet, and resuspending the pellet with DMEM containing a 5-10% human serum or a 5-10% fetal bovine serum to obtain the human scalp hair follicle single cell suspension.

9. A human scalp hair follicle single cell suspension prepared by the preparation method according to claim 1.

10. A method of an application of the human scalp hair follicle single cell suspension according to claim 9 for a preparation of a medicament to treat a vitiligo.

11. The human scalp hair follicle single cell suspension according to claim 9, wherein in step 1, the low temperature environment is 2-8° C.; and a treatment time of the pretreating with the digestive enzyme solution is 1-1.5 hours.

12. The human scalp hair follicle single cell suspension according to claim 9, wherein in step 1 and step 2, a mass percent concentration of the digestive enzyme solution is 0.5-1.5%; the digestive enzyme solution is prepared according to the following formula: evenly mixing a dispase solution with a mass percentage concentration of 1%-3% and a collagenase solution with a mass percentage concentration of 1%-3% in equal volumes to obtain the digestive enzyme solution.

13. The human scalp hair follicle single cell suspension according to claim 9, wherein the following steps are added before step 1: substep 1, a human scalp pretreatment: in a sterile environment, cutting off an excess fat from an isolated scalp tissue block to obtain a resulting scalp tissue block, and cutting the resulting scalp tissue block into blocks with a size of 0.1-0.2 cm², and substep takes 3-5 minutes; substep 2, carrying out a disinfection treatment on the blocks to obtain the fresh isolated scalp tissue, and a disinfection treatment time is 9-21 minutes.

14. The human scalp hair follicle single cell suspension according to claim 13, wherein the disinfection treatment comprises: placing the blocks in PBS with antibiotics, washing the blocks twice for 5 minutes each time, and finally washing the blocks once with PBS without antibiotics for 5 minutes; the PBS with antibiotics comprises 400 u/ml penicillin, 400 ug/ml streptomycin, and 10 ug/ml amphotericin B.

15. The human scalp hair follicle single cell suspension according to claim 9, wherein step 1 comprises: adding a pre-cooled 0.5-1.5% digestive enzyme solution into a clean and sterile tube to submerge the fresh isolated scalp tissue, wherein if the fresh isolated scalp tissue is too much, a volume of the pre-cooled 0.5-1.5% digestive enzyme solution is subject to completely submerge the fresh isolated scalp tissue, and standing the pre-cooled 0.5-1.5% digestive enzyme solution submerging the fresh isolated scalp tissue at 2-8° C. for 1-1.5 hours.

16. The human scalp hair follicle single cell suspension according to claim 9, wherein step 2 comprises: incubating and digesting the digestive enzyme solution by shaking at 100-180 rpm at 30-37° C. for 30 minutes to 1 hour, and the pretreated scalp tissue are transferred to a culture dish with DMEM to dissociate the hair follicles.

17. The human scalp hair follicle single cell suspension according to claim 9, wherein a digestion time in step 3 is 15-30 minutes, and step 3 comprises: transferring the dissociated hair follicles to 0.25%-0.5% pancreatin-EDTA to completely submerge the dissociated hair follicles, standing the 0.25%-0.5% pancreatin-EDTA submerging the dissociated hair follicles for the digesting for 15-30 minutes at 30-37° C. to obtain a digested solution, then adding DMEM and a human serum or a fetal bovine serum in the digested solution to make up a volume of a resulting mixture to 5-10 ml, wherein a final concentration of the human serum or the fetal bovine serum is 5-10%, mixing the resulting mixture well and then pipetting the resulting mixture, a number of the pipetting does not exceed 30 times; removing a hair rod from the resulting mixture to obtain a remaining liquid, centrifuging the remaining liquid at 800-1200 rpm for 5-10 minutes at room temperature to obtain a centrifuged remaining liquid, and then removing a supernatant from the centrifuged remaining liquid to obtain a pellet, and resuspending the pellet with DMEM containing a 5-10% human serum or a 5-10% fetal bovine serum to obtain the human scalp hair follicle single cell suspension.
